(19) United States
(12) Patent Application Publication
(10) Pub. No.: US 2010/0105927 A1
(43) Pub. Date: Apr. 29, 2010

(54) POLYNARY VANADYL PYROPHOSPHATE

(75) Inventors: Hartmut Hibst, Schriesheim (DE); Robert Glaum, Rheinbach-Flerzheim (DE); Ernst Benser, Bonn (DE)

Correspondence Address: CONNOLLY BOVE LODGE & HUTZ LLP, P.O. BOX 2207, WILMINGTON, DE, 19899, US

(73) Assignee: BASF SE, Ludwigshafen (DE)

(21) Appl. No.: 12/531,485

(22) PCT Filed: Mar. 12, 2008
(86) PCT No.: PCT/EP08/52948

§ 371 Date: Sep. 15, 2009

(30) Foreign Application Priority Data
Mar. 16, 2007 (DE) 10 2007 012 723.7

(51) Int. Cl. C07D 307/60; B01J 27/198

(52) U.S. Cl. 549/256; 502/209

(57) ABSTRACT

A novel polynary vanadyl pyrophosphate of the general formula I

(VO)ₐ(M_{1-b}V_{b})₂(P₂O₇)_{c}

is described, in which M is one or more metals selected from Ti, Zr, Hf, Cr, Fe, Co, Ni, Ru, Rh, Pd, Cu, Zn, B, Al, Ga and In, a is from 0.5 to 1.5, b is from 0 to 0.9, c is from 1.5 to 2.5, having a crystal structure whose powder X-ray diffractogram is characterized by defined reflections. A preferred representative is (VO)Fe₂(P₂O₇)₂. The vanadyl pyrophosphates are suitable as gas phase oxidation catalysts, for example for preparing maleic anhydride from a hydrocarbon having at least four carbon atoms.

The present invention relates to a polynary vanadyl pyrophosphate, to a process for its preparation and to its use for heterogeneously catalyzed gas phase oxidations, preferably heterogeneously catalyzed gas phase oxidations of a hydrocarbon having at least four carbon atoms.

Heterogeneous catalysts based on vanadyl pyrophosphate (VO)₂P₂O₇ (so-called VPO catalysts) are used in the industrial oxidation of n-butane to maleic anhydride, and also in a series of further oxidation reactions of hydrocarbons.

The vanadyl pyrophosphate catalysts are generally prepared as follows: (1) synthesis of a vanadyl hydrogenphosphate hemihydrate precursor (VOHPO₄.½H₂O) from a pentavalent vanadium compound (e.g. V₂O₅), a penta- or trivalent phosphorus compound (e.g. ortho- and/or pyrophosphoric acid, phosphoric esters or phosphorous acid) and a reducing alcohol (e.g. isobutanol), isolation of the precipitate, drying and optionally shaping (e.g. tableting) and (2) preforming the precursor to vanadyl pyrophosphate ((VO)₂P₂O₂) by calcining. Reference is made, for example, to EP-A 0 520 972 and WO 00/72963.

As a result of the use of an alcohol as a reducing agent, generally several % by weight of organic compounds remain included in the precursor and cannot be removed even by careful washing. In the further catalyst preparation, especially in the calcination, these exert an adverse effect on the catalytic properties of the catalyst. For instance, in the subsequent calcination, the risk exists of evaporation or of thermal decomposition of this included organic compound to form gaseous components which can lead to a pressure rise in the interior of the crystals and hence to destruction of the catalyst structure. This adverse effect is particularly marked in the case of calcination under oxidizing conditions, since the formation of the oxidized by-products, for example carbon monoxide or carbon dioxide, forms a significantly greater amount of gas. Furthermore, the oxidation of these organic compounds forms locally very large amounts of heat which can lead to thermal damage of the catalyst.

Moreover, the included organic compounds also have a significant influence on the adjustment of the local oxidation state of the vanadium. For instance, B. Kubias et al. in Chemie Ingenieur Technik 72 (3), 2000, pages 249-251 demonstrate the reducing effect of organic carbon in anaerobic calcination (under nonoxidizing conditions) of a vanadyl hydrogenphosphate hemihydrate precursor obtained from isobutanolic solution. In the example mentioned, anaerobic calcination afforded a mean oxidation state of the vanadium of 3.1, whereas aerobic calcination (under oxidizing conditions) afforded a mean oxidation state of the vanadium of about 4.

To improve the catalytic performance, it has been proposed to add small amounts of oxides of di-, tri- or tetravalent transition metals, known as promoters, to the vanadyl pyrophosphate (cf. G. J. Hutchings, J. Mater. Chem. 2004, 14, 3385-3395; K. V. Narayana et al., Z. Anorg. Allg. Chem. 2005, 631, 25-30). The mode of action of these promoters is to date substantially unexplained.

The literature to date does not include any information about the existence and the catalytic behavior of monophasic polynary vanadium(IV) phosphates which comprise a di-, tri- or tetravalent transition metal other than vanadium.

A mixed-valency vanadium(III,IV) diphosphate, V^{III}₂(V^{IV}O)(P₂O₇)₂, has already been known for some time and also characterized by crystallographic means; cf. J. W. Johnson et al., Inorg. Chem. 1988, 27, 1646-1648. B. G. Golovkin, V. L. Volkov, Russ. J. Inorg. Chem. 1987, 32, 739-741 discloses a further compound which has likewise been described as the diphosphate V₃O₄(P₂O₇); however, there is a complete lack of information on its characterization.

It was an object of the present invention to provide novel polynary vanadyl pyrophosphates.

It was a further object of the present invention to provide novel polynary vanadyl pyrophosphates with catalytic properties for heterogeneously catalyzed gas phase oxidations.

It was a further object of the present invention to provide novel polynary vanadyl pyrophosphates with whose aid the catalytic properties of known heterogeneous catalysts based on vanadyl pyrophosphate can be modified.

Further objects of the invention related to the provision of processes for preparing the novel polynary vanadyl pyrophosphates and processes for heterogeneously catalyzed gas phase oxidation.

Accordingly, a novel polynary vanadyl pyrophosphate of the general formula I

(VO)ₐ(M_{1-b}V_{b})₂(P₂O₇)_{c}

has been found, in which
M is one or more metals selected from Ti, Zr, Hf, Cr, Fe, Co, Ni, Ru, Rh, Pd, Cu, Zn, B, Al, Ga and In,
a is from 0.5 to 1.5,
b is from 0 to 0.9,
c is from 1.5 to 2.5,
   having a crystal structure whose powder X-ray diffractogram is characterized by the presence of at least 7, preferably all, of the following 10 reflections at the interplanar spacings d[Å]=7.11±0.06, 4.20±0.04, 4.12±0.04, 3.61±0.04, 3.56±0.04, 3.23±0.04, 3.01±0.04, 2.98±0.04, 2.52±0.04, 2.51±0.04.

In this application, the X-ray reflections are reported in the form of the interplanar spacings d [Å] which are independent of the wavelength of the X-radiation used. The wavelength λ of the X-radiation used for diffraction and the diffraction angle θ (in this document, the reflection position used is the peak location of a reflection in the 20 plot) are linked to one another via the Bragg equation as follows:

2 sin θ=λ/d

where d is the interplanar spacing of the atomic three-dimensional arrangement corresponding to the particular reflection.

The powder X-ray diffractogram of the inventive polynary vanadyl pyrophosphate of the formula I is characterized by the reflections listed above. The reflections generally have the approximate relative intensities (Iᵣₑₗ [%]) specified in Table 1. Further, generally less intensive reflections of the powder X-ray diffractogram have not been included in Table 1.

TABLE 1

| | Rel. intensity [%] |
|---|---|
| 7.11 ± 0.06 | 25 ± 10 |
| 4.20 ± 0.04 | 100 |
| 4.12 ± 0.04 | 30 ± 20 |
| 3.61 ± 0.04 | 50 ± 40 |
| 3.56 ± 0.04 | 25 ± 23 |
| 3.23 ± 0.04 | 20 ± 15 |
| 3.01 ± 0.04 | 35 ± 25 |
| 2.98 ± 0.04 | 40 ± 30 |
| 2.52 ± 0.04 | 50 ± 40 |
| 2.51 ± 0.04 | 35 ± 30 |

Depending on the crystallinity and the texture of the resulting crystals of the inventive polynary vanadyl pyrophosphate, however, there may be enhancement or attenuation of the intensity of the reflections in the powder X-ray diffractogram. The attenuation may be to such an extent that individual reflections in the powder X-ray diffractogram are no longer detectable.

It is self-evident to the person skilled in the art that mixtures of the inventive polynary vanadyl pyrophosphates with other crystalline compounds have additional reflections. Such mixtures of the polynary vanadyl pyrophosphate with other crystalline compounds can be prepared in a controlled manner by mixing the inventive polynary vanadyl pyrophosphate or can be formed in the preparation of the inventive polynary vanadyl pyrophosphates by incomplete conversion of the starting materials or formation of extraneous phases with different crystal structure.

In the formula I, a is preferably from 0.8 to 1.2, especially about 1.

In formula I, b is preferably from 0 to 0.4. In certain inventive embodiments, b is 0.

In formula I, c is preferably from 1.8 to 2.2, especially about 2.

In formula I, M is a metal selected from Ti, Zr, Hf, Cr, Fe, Co, Ni, Ru, Rh, Pd, Cu, Zn, B, Al, Ga and In, or combinations of two or more of these metals. M is preferably a metal selected from Cr and Fe.

A preferred inventive polynary vanadyl pyrophosphate has the following formula:

(VO)Fe₂(P₂O₇)₂.

The inventive polynary vanadyl pyrophosphates are obtainable in various ways.

Firstly, the inventive polynary vanadyl pyrophosphates can be obtained by a solid-state reaction in a closed system. For this purpose, at least two reactants selected from oxygen compounds of vanadium, phosphorus compounds of vanadium and mixed oxygen-phosphorus compounds of vanadium, elemental vanadium, oxygen compounds of the metal M, phosphorus compounds of the metal M and mixed oxygen-phosphorus compounds of the metal M and elemental metal M are reacted.

In this case, the reactants are generally selected such that (i) they provide the desired stoichiometry of the elements in the formula I and (ii) the sum of the products of valency multiplied by frequency of the elements other than oxygen in the reactants corresponds to the sum of the products of valency multiplied by frequency of the elements other than oxygen in the formula I. The starting compounds may be selected such that all elements other than oxygen therein already possess the valency that they possess in the formula I. Alternatively, the starting compounds can be selected such that some or all elements other than oxygen therein possess a valency different from that which they possess in formula I. As a result of redox reactions, for example a synproportionation, during the solid-state reaction, the elements other than oxygen receive the valency which they possess in the formula I. For example, it is possible to use a combination of equivalent amounts of vanadium(III) and vanadium(V) compounds from which tetravalent vanadium forms in the solid-state reaction.

The starting compounds required, in the form of oxides, phosphates, oxide phosphates, phosphides or the like, are either commercially available or known from the literature or can be synthesized easily by the person skilled in the art in analogy to known preparation methods.

The starting materials are mixed intimately, for example by fine trituration. The solid-state reaction is effected typically at a temperature of at least 500° C., for example from 650 to 1100° C., especially about 800° C. Typical reaction times are, for example, from 24 hours to 10 days. Suitable reaction vessels consist, for example, of quartz glass or corundum.

In order to obtain products with a high crystallinity or single crystals, it is appropriately possible in the solid-state reaction to use a suitable mineralizer, such as iodine or PtCl₂.

Alternatively, inventive polynary vanadyl pyrophosphates can be prepared by
a) preparing a dry mixture of a vanadium source, of a source of the metal M and of a phosphate source,
b) optionally providing reduction equivalents in order to convert the vanadium and/or the metal M to the valency state possessed by the vanadium and the metal M in the formula I and
c) calcining the dry mixture at least 500° C.

To this end, a mixture of suitable sources of the elemental constituents of the inventive polynary vanadyl pyrophosphates is used to obtain a very intimate, preferably finely divided, dry mixture of the desired constituent stoichiometry.

The starting compounds can be mixed intimately in dry or in wet form.

When it is effected in dry form, the starting compounds are appropriately used as finely divided powders and, after the mixing and optionally compaction, subjected to calcination (thermal treatment).

However, preference is given to effecting the intimate mixing in wet form, i.e. in dissolved or suspended form. The starting compounds are typically mixed with one another in the form of an aqueous solution (optionally with use of complexing agents) and/or suspension. Subsequently, the aqueous solution or suspension is dried and, after the drying, calcined.

The drying can be effected by evaporation under reduced pressure, by freeze-drying or by conventional evaporation. However, preference is given to effecting the drying process by spray-drying. The exit temperatures are generally from 70 to 150° C.; the spray-drying can be performed in cocurrent or in countercurrent.

Suitable vanadium sources are, for example, vanadyl sulfate hydrate, vanadyl acetylacetonate, vanadates such as ammonium metavanadate, vanadium oxides, for example divanadium pentoxide (V₂O₅), vanadium dioxide (VO₂) or divanadium trioxide (V₂O₃), vanadium halides, for example vanadium tetrachloride (VCl₄) and vanadyl halides, for example VOCl₃. Divanadium pentoxide and ammonium vanadate are preferred vanadium sources.

Useful sources for the metal M include all compounds of the elements which are capable of forming oxides and/or hydroxides when heated (optionally in the presence of molecular oxygen, for example under air). Of course, the starting compounds of this type which are used may also partly or exclusively already be oxides and/or hydroxides of the elemental constituents. The source of the metal M is preferably selected from nitrates, carboxylates, carbonates, hydrogencarbonates, basic carbonates, oxides, hydroxides and oxide hydroxides of the metal M.

Suitable phosphate sources are compounds comprising phosphate groups or compounds from which phosphate groups form by redox reactions and/or in the course of heating (optionally in the presence of molecular oxygen, for example under air). These include phosphoric acids, especially orthophosphoric acid, pyro- or metaphosphoric acids, phosphorous acid, hypophosphorous acid, phosphates or hydrogenphosphates such as diammonium hydrogenphosphate, and elemental phosphorus, for example white phosphorus. The phosphate source is preferably formed at least partly by phosphorous acid or hypophosphorous acid, optionally in combination with orthophosphoric acid.

When the vanadium sources or sources for the metal M used are compounds in which the vanadium or the metal M has a higher valency than it possesses in the formula I (i.e. than the formal valency of V and M which is required to obtain electrical neutrality with the O²⁻ and PO₄³⁻ anions present in the formula I), reduction equivalents should preferably be provided in order to convert the vanadium and/or the metal M to the valency state that the vanadium and the metal M possess in the formula I.

The reduction equivalents are provided by a reducing agent which is capable of reducing the higher-valency form of the vanadium or of the metal M. The reduction can be effected in the course of preparation of the dry mixture or in the course of calcination at the latest. Preference is given to preparing the intimate thy mixture under inert gas atmosphere (e.g. N₂) in order to ensure better control over the oxidation states.

Preferred reducing agents for this purpose are selected from hypophosphorous acid, phosphorous acid, hydrazine (as the free base or hydrate or in the form of its salts such as hydrazine dihydrochloride, hydrazine sulfate), hydroxylamine (as the free base or in the form of its salts such as hydroxylamine hydrochloride), nitrosylamine, elemental vanadium, elemental phosphorus, borane (including in the form of complex borohydrides such as sodium borohydride) or oxalic acid. Phosphorous acid and/or hypophosphorous acid are preferred reducing agents.

It is self-evident that particular reducing agents such as hypophosphorous acid or phosphorous acid can simultaneously serve as the phosphate source, or elemental vanadium simultaneously serves as the vanadium source.

The dry mixture is treated thermally at temperatures of at least 500° C., preferably from 700 to 1000° C., especially about 800° C. The thermal treatment can be effected under an oxidizing, reducing or inert atmosphere. Useful oxidizing atmosphere includes, for example, air, air enriched with molecular oxygen or air depleted of oxygen. However, preference is given to performing the thermal treatment under inert atmosphere, i.e., for example, under molecular nitrogen and/or noble gas. The thermal treatment is typically effected at standard pressure (1 atm). Of course, the thermal treatment can also be effected under reduced pressure or under elevated pressure.

When the thermal treatment is effected under gaseous atmosphere, the latter may either be stationary or flow. It preferably flows. Overall, the thermal treatment may take up to 24 h or more.

The invention further relates to a gas phase oxidation catalyst which comprises at least one inventive polynary vanadyl pyrophosphate. The polynary vanadyl pyrophosphates may be used as such, for example as powders, or in the form of shaped bodies as heterogeneous catalysts.

Preference is given to effecting the shaping by tableting. For tableting, a tableting assistant is generally added to the powder and mixed intimately.

Tableting assistants are generally catalytically inert and improve the tableting properties of the powder, for example by increasing the lubrication and free flow. Suitable and preferred tableting assistants include graphite or boron nitride. The tableting assistants added generally remain in the activated catalyst.

The powder can also be tableted and subsequently comminuted to spall.

The shaping to shaped bodies can, for example, also be effected by applying at least one inventive polynary vanadyl pyrophosphate or mixtures which comprise at least one inventive polynary vanadyl pyrophosphate to a support body.

The support bodies are preferably chemically inert. In other words, they essentially do not intervene in the course of the catalytic gas phase oxidation which is catalyzed by the inventive polynaryl vanadyl pyrophosphates.

Useful materials for the support bodies include especially aluminum oxide, silicon dioxide, silicates such as clay, kaolin, steatite, pumice, aluminum silicate and magnesium silicate, silicon carbide, zirconium dioxide and thorium dioxide.

The surface of the support body may either be smooth or rough. Advantageously, the surface of the support body is rough, since an increased surface roughness generally causes an increased adhesion strength of the applied active composition coating.

Moreover, the support material may be porous or nonporous. The support material is appropriately nonporous, i.e. the total volume of the pores is preferably less than 1% by volume, based on the volume of the support body.

The thickness of the catalytically active layer is typically from 10 to 1000 μm, for example from 50 to 700 μm, from 100 to 600 μm or from 150 to 400 μm.

In principle, support bodies with any geometric structure are useful. Their longest dimension is generally from 1 to 10 mm. However, preference is given to employing spheres or cylinders, especially hollow cylinders, as support bodies.

In the simplest manner, the coated catalysts can be prepared by preforming a mass of a polynary vanadyl pyrophosphate of the general formula (I), converting it to finely divided form and finally applying it to the surface of the support body with the aid of a liquid binder. To this end, the surface of the support body is, in the simplest manner, moistened with the liquid binder, and a layer of the active composition is adhered on the moistened surface by contacting it with the finely divided material. Finally, the coated support body is dried. Of course, the operation can be repeated to achieve a greater layer thickness.

The inventive polynary vanadyl pyrophosphates may also be used in order to modify the catalytic properties, especially conversion and/or selectivity, of known catalysts, especially based on vanadyl pyrophosphate. To this end, the inventive polynary vanadyl pyrophosphates may be used, for example, as a promoter phase in a catalyst based on vanadyl pyrophosphate. Appropriately, the catalyst then comprises a first phase and a second phase in the form of three-dimensional regions which are delimited from their local environment by a different chemical composition. In this case, the first phase comprises a catalytically active material based on vanadyl pyrophosphate and the second phase at least one inventive polynary vanadyl pyrophosphate. In this case, (i) finely divided particles of the second phase may be dispersed in the first phase, or (ii) the first phase and the second phase may be distributed relative to one another as in a mixture of finely divided first phase and finely divided second phase.

These biphasic catalysts can be prepared, for example, by preparing a vanadyl hydrogenphosphate hemihydrate precursor (VOHPO₄.½H₂O), admixing it with preformed particles of the second phase of inventive polynary vanadyl pyrophosphate, shaping the resulting material and calcining it. The vanadyl hydrogenphosphate hemihydrate precursor can be obtained in a manner known per se from a compound of pentavalent vanadium (e.g. V₂O₅), a compound comprising penta- or trivalent phosphorus (e.g. ortho- and/or pyrophosphoric acid, phosphoric ester or phosphorous acid) and a reducing alcohol (e.g. isobutanol), and isolating the precipitate. Reference is made, for example, to EP-A 0 520 972 and WO 00/72963.

The inventive catalysts whose catalytically active composition comprises at least one above-defined polynary vanadyl pyrophosphate may also be combined with catalysts based on vanadyl pyrophosphate in the form of a structured packing. For instance, a gas stream which comprises a hydrocarbon to be oxidized and molecular oxygen can be passed through a bed of a first gas phase oxidation catalyst placed upstream in flow direction of the gas stream and then through one or more downstream beds of a second or further gas phase oxidation catalysts, in which case the first or second or one of the further beds comprises an inventive catalyst.

The invention further relates to a process for partial gas phase oxidation or ammoxidation, in which a gas stream which comprises a hydrocarbon and molecular oxygen is contacted with an inventive catalyst. In the case of ammoxidation, the gas stream additionally comprises ammonia. In the context of the present invention, ammoxidation is understood to mean a heterogeneously catalyzed process in which methyl-substituted alkenes, arenes and hetarenes are converted to nitriles by reaction with ammonia and oxygen in the presence of transition metal catalysts.

In preferred embodiments, the process for partial gas phase oxidation serves to prepare maleic anhydride, in which case the hydrocarbon used comprises at least four carbon atoms.

In the process according to the invention for partial gas phase oxidation or ammoxidation, generally tube bundle reactors are used. Alternatively, it is also possible to use fluidized bed reactors.

Suitable hydrocarbons are generally aliphatic and aromatic, saturated and unsaturated hydrocarbons having at least four carbon atoms, for example 1,3-butadiene, 1-butene, cis-2-butene, trans-2-butene, n-butane, C₄ mixtures, 1,3-pentadiene, 1,4-pentadiene, 1-pentene, cis-2-pentene, trans-2-pentene, n-pentane, cyclopentadiene, dicyclopentadiene, cyclopentene, cyclopentane, C₅ mixtures, hexenes, hexanes, cyclohexane and benzene. Preference is given to using 1,3-butadiene, 1-butene, cis-2-butene, trans-2-butene, n-butane, benzene or mixtures thereof.

Particular preference is given to the use of n-butane and n-butane-containing gases and liquids.

The n-butane used may stem, for example, from natural gas, from steam crackers or FCC crackers.

The hydrocarbon is generally added under quantitative control, i.e. with constant specification of a defined amount per unit time. The hydrocarbon can be metered in in liquid or gaseous form.

Preference is given to metered addition in liquid form with subsequent evaporation before entry into the reactor.

The oxidizing agents used are oxygen-comprising gases, for example air, synthetic air, a gas enriched with oxygen or else so-called “pure” oxygen, i.e. oxygen stemming, for example, from air fractionation. The oxygen-comprising gas is preferably also added under quantitative control.

The gas to be passed through the reactor generally comprises a hydrocarbon concentration of from 0.5 to 15% by volume and an oxygen concentration of from 8 to 25% by volume. The proportion lacking from 100% by volume is composed of further gases, for example nitrogen, noble gases, carbon monoxide, carbon dioxide, steam, oxygenated hydrocarbons (e.g. methanol, formaldehyde, formic acid, ethanol, acetaldehyde, acetic acid, propanol, propionaldehyde, propionic acid, acrolein, crotonaldehyde) and mixtures thereof. In the case of selective oxidation of n-butane, the n-butane content in the total amount of hydrocarbon is preferably more than 90% and more preferably more than 95%.

To ensure a long catalyst lifetime and further increase in conversion, selectivity, yield, catalyst hourly space velocity and space-time yield, a volatile phosphorus compound is preferably added to the gas in the process according to the invention.

At the start, i.e. at the reactor inlet, its concentration is at least 0.2 ppm by volume, i.e. 0.2·10⁻⁶ parts by volume of the volatile phosphorus compounds based on the total volume of the gas at the reactor inlet. Preference is given to a content of from 0.2 to 20 ppm by volume, particular preference to a content of from 0.5 to 10 ppm by volume.

Volatile phosphorus compounds are understood to mean all of those phosphorus-comprising compounds which are present in gaseous form under the use conditions in the desired concentration. Examples of suitable volatile phosphorus compounds include phosphines and phosphoric esters. Particular preference is given to the C₁- to C₄-alkyl phosphates, very particular preference to trimethyl phosphate, triethyl phosphate and tripropyl phosphate, especially triethyl phosphate.

The process according to the invention is performed generally at a temperature of from 300 to 500° C. The temperature mentioned is understood to mean the temperature of the catalyst bed present in the reactor which would be present when the process is executed in the absence of a chemical reaction.

When this temperature is not exactly the same at all points, the term means the numerical average of the temperatures along the reaction zone. In particular, this means that the true temperature present over the catalyst, owing to the exothermicity of the oxidation reaction, may also be outside the range mentioned. Preference is given to performing the process according to the invention at a temperature of from 380 to 460° C., more preferably from 380 to 430° C.

The process according to the invention can be executed at a pressure below standard pressure (for example up to 0.05 MPa abs) or else above standard pressure (for example up to 10 MPa abs). This is understood to mean the pressure present in the reactor unit. Preference is given to a pressure of from 0.1 to 1.0 MPa abs, particular preference to a pressure of from 0.1 to 0.5 MPa abs.

The process according to the invention can be performed in two preferred process variants, the variant with “straight pass” and the variant with “recycling”. In “straight pass”, maleic anhydride and any oxygenated hydrocarbon by-products are removed from the reactor effluent and the remaining gas mixture is discharged and optionally utilized thermally. In the case of “recycling”, maleic anhydride and any oxygenated hydrocarbon by-products are likewise removed from the reactor effluent, the remaining gas mixture which comprises unconverted hydrocarbon is recycled fully or partly to the reactor. A further variant of “recycling” is the removal of the unconverted hydrocarbon and the recycling thereof to the reactor.

In a particularly preferred embodiment for preparation of maleic anhydride, n-butane is used as the starting hydrocarbon and the heterogeneously catalyzed gas phase oxidation is performed in “straight pass” over the inventive catalyst.

The present invention is illustrated in detail by the appended figures and the examples which follow.

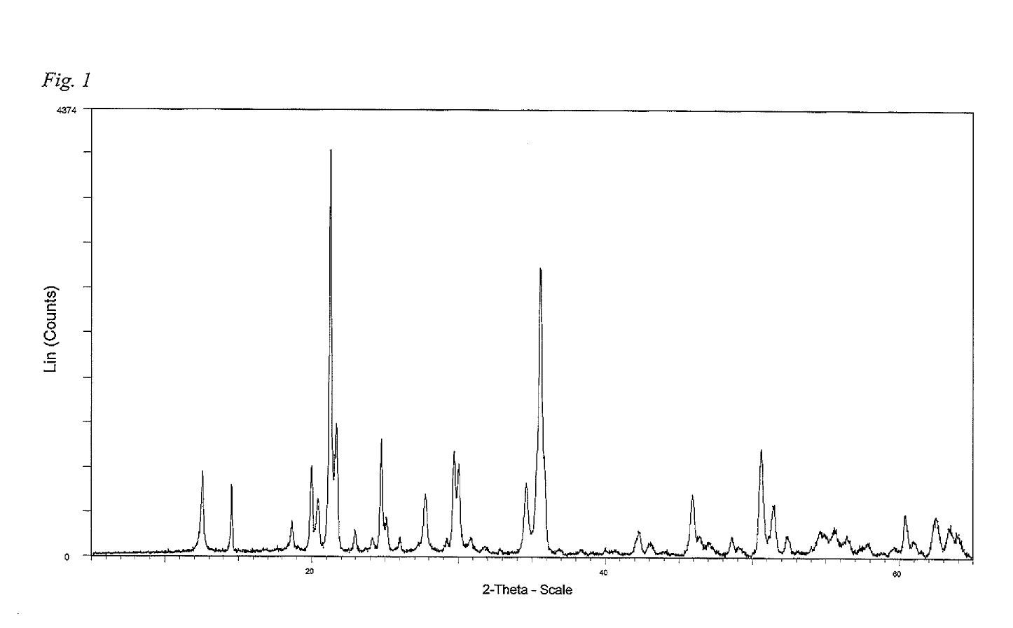

FIG. 1 shows the powder X-ray diffractogram of (VO)Fe₂(P₂O₇)₂;

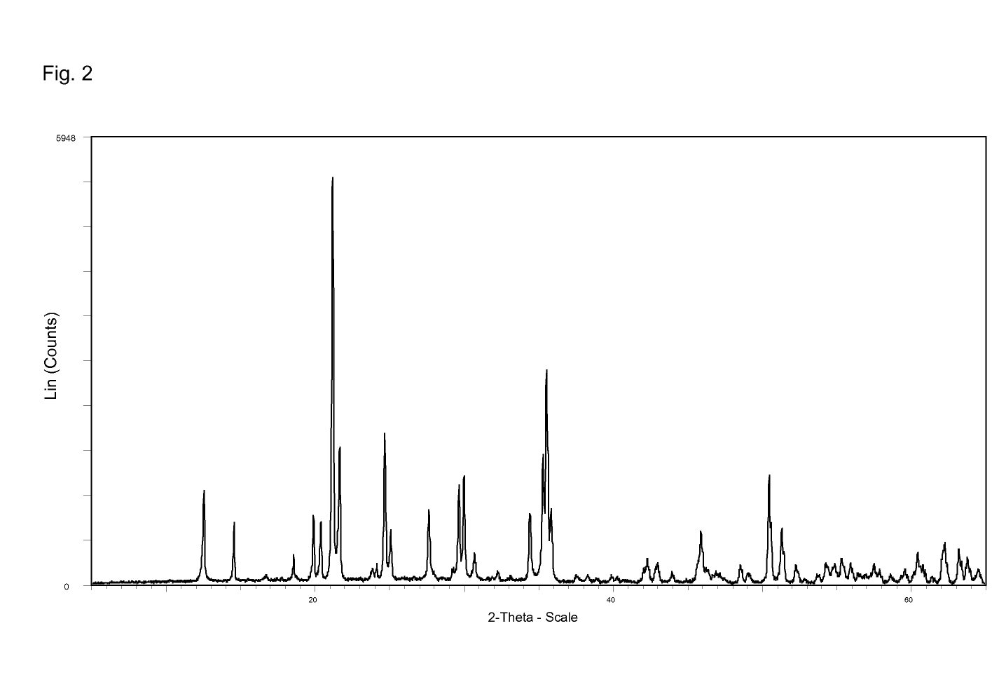

FIG. 2 shows the powder X-ray diffractogram of (VO)Fe₂(P₂O₇)₂ which has been prepared by an alternative process.

The X-ray diffraction analyses are derived from X-ray diffractograms obtained using Cu—Kα radiation (λ=1.54178 Å) as the X-radiation (Siemens Theta-Theta D-5000 diffractometer, tube voltage: 40 kV, tube current: 40 mA, aperture V20 (variable) collimator V20 (variable), secondary monochromator aperture (0.1 mm), detector aperture (0.6 mm), measurement interval (2θ): 0.02 [°], measurement time per step: 2.4 s, detector: scintillation counting tube).

### Example 1

### Preparation of (VO)Fe₂(P₂O₇)₂

The title compound was obtained according to the following empirical equation:

Fe(NO₃)₃.9H₂O+NH₄VO₃+0.5H₃PO₃+3.5H₃PO₄→(VO)Fe₂(P₂O₇)₂+NH₄NO₃+5HNO₃+21.5H₂O

In a glass reactor, a mixture of 6.0 l of water, 808.0 g of Fe(NO₃)₃.9H₂O (Sigma Aldrich, Seelze, Germany), 117.0 g of NH₄VO₃ (with a V₂O₅ content of 77.57% (=0.5 mol of V), H. C, Starck GmbH, Goslar, Germany), 403.5 g of H₃PO₄ (85% (=3.5 mol of P), Sigma Aldrich, Seelze, Germany) and 82.0 g of H₃PO₃ (50% (=0.5 mol of P), Sigma Aldrich, Seelze, Germany) was heated to 90° C. with stirring and stirred at this temperature for 2 hours. The resulting suspension was dried in a spray-dryer (Mobile Minor™ 2000, MM, from Niro AIS, Soborg, Denmark, entrance temperature: 330° C., exit temperature: 107° C.) under nitrogen. The resulting spray powder was calcined at 800° C. in a nitrogen atmosphere in a rotary quartz tube with an internal volume of 1 l for two hours.

The resulting powder had a specific BET surface area of 3.5 m²/g. The following 2θ values with the accompanying intensities I and interplanar spacings d were determined from the powder X-ray diffractogram (FIG. 1).

| 2θ | I | d [Å] |
|---|---|---|
| 12.44° | 21% | 7.11 |
| 21.18° | 100% | 4.19 |
| 21.60° | 33% | 4.11 |
| 24.66° | 29% | 3.61 |
| 25.03° | 9% | 3.55 |
| 27.70° | 15% | 3.22 |
| 29.64° | 26% | 3.01 |
| 29.96° | 23% | 2.98 |
| 35.54° | 71% | 2.52 |
| 35.84° | 23% | 2.50 |

### Example 2

### Alternative Preparation of (VO)Fe₂(P₂O₇)₂

The title compound was obtained according to the following empirical equation:

2FeOOH+0.5V₂O₅+0.5H₃PO₃+3.5H₃PO₄→(VO)Fe₂(P₂O₇)₂+7H₂O

In a glass reactor, a mixture of 6.0 l of water, 90.9 g of V₂O₅ [>99%, 0.5 mol, calculated as V] (GfE Umwelttechnik GmbH, Nuremburg, Germany), 187.1 g of FeOOH [95%, 2.0 mol, calculated as Fe] (Sicopur Gelb, BASF Aktiengesellschaft, Ludwigshafen, Germany), 403.5 g of H₃PO₄ (85% (=3.5 mol of P), Sigma Aldrich, Seelze, Germany) and 82.0 g of H₃PO₃ (50% (=0.5 mol of P), Sigma Aldrich, Seelze, Germany) was heated to 90° C. with stirring and stirred at this temperature for 2 hours. The resulting suspension was dried in a spray dryer (Mobile Minor™ 2000, MM, from Niro AIS, Soborg, Denmark, entrance temperature: 330° C., exit temperature: 107° C.) under nitrogen. The resulting spray powder was calcined in two stages: first, it was calcined at 600° C. in a nitrogen atmosphere in a rotary quartz tube with an internal volume of 1 l for two hours. The product was ground in a ball mill for 15 min and calcined at 850° C. in a nitrogen atmosphere for a further two hours.

The resulting powder had a specific BET surface area of 1.6 m²/g. The following 2θ values with the accompanying intensities and the interplanar spacings d were determined from the powder X-ray diffractogram (FIG. 2).

| 2θ | I | d [Å] |
|---|---|---|
| 12.44° | 23% | 7.11 |
| 21.11° | 100% | 4.20 |
| 21.58° | 34% | 4.12 |
| 24.63° | 37% | 3.61 |
| 25.03° | 13% | 3.56 |
| 27.60° | 18% | 3.23 |
| 29.62° | 24% | 3.01 |
| 29.95° | 27% | 2.98 |
| 35.54° | 53% | 2.52 |
| 35.84° | 18% | 2.51 |

1. A polynary vanadyl pyrophosphate of the general formula I
(VO)ₐ(M_{1-b}V_{b})₂(P₂O₇)_{c}
in which
M is one or more metals selected from the group consisting of: Ti, Zr, Hf, Cr, Fe, Co, Ni, Ru, Rh, Pd, Cu, Zn, B, Al, Ga and In,
a is from 0.5 to 1.5,
bis from 0 to 0.9, and
c is from 1.5 to 2.5,
wherein the polynary vanadyl pyrophosphate has a crystal structure having a powder X-ray diffractogram is characterized by the presence of at least 7 of the following 10 reflections at the interplanar spacings d [Å]=7.11±0.06, 4.20±0.04, 4.12±0.04, 3.61±0.04, 3.56±0.04, 3.23±0.04, 3.01±0.04, 2.98±0.04, 2.52±0.04, 2.51±0.04.

2. The vanadyl pyrophosphate of claim 1, wherein the reflections have the following relative intensities:

| d [Å] | Rel. intensity [%] |
|---|---|
| 7.11 ± 0.06 | 25 ± 10 |
| 4.20 ± 0.04 | 100 |
| 4.12 ± 0.04 | 30 ± 20 |
| 3.61 ± 0.04 | 50 ± 40 |
| 3.56 ± 0.04 | 25 ± 23 |
| 3.23 ± 0.04 | 20 ± 15 |
| 3.01 ± 0.04 | 35 ± 25 |
| 2.98 ± 0.04 | 40 ± 30 |
| 2.52 ± 0.04 | 50 ± 40 |
| 2.51 ± 0.04 | 35 ± 30 |

3. The vanadyl pyrophosphate of claim 1, in which
a is from 0.8 to 1.2,
b is from 0 to 0.4, and
c is from 1.8 to 2.2.

4. The vanadyl pyrophosphate of claim 1, M is Fe or Cr.

5. The vanadyl pyrophosphate of claim 4 having the formula (VO)Fe₂(P₂O₇)₂.

6. A process for preparing the polynary vanadyl pyrophosphate of claim 1 comprising:
selecting at least two reactants selected from the group consisting of: oxygen compounds of vanadium, phosphorus compounds of vanadium and mixed oxygen-phosphorus compounds of vanadium, elemental vanadium, oxygen compounds of the metal M, phosphorus compounds of the metal M and mixed oxygen-phosphorus compounds of the metal M and elemental metal M; and
allowing the selected reactants to react in a sold-state reaction in a closed system.

7. A process for preparing the polynary vanadyl pyrophosphate of claim 1 comprising:
preparing a dry mixture comprising a vanadium source and a phosphate source, and
calcining the dry mixture at a temperature of at least 500° C.

8. The process of claim 20, wherein the reduction equivalents are provided by a reducing agent which is selected from the group consisting of: hypophosphorous acid, phosphorous acid, hydrazine, hydroxylamine, nitrosylamine, elemental vanadium, elemental phosphorus, borane and oxalic acid.

9. The process of claim 19, wherein the dry mixture is prepared by mixing the vanadium source, the source of the metal M, the phosphate source and a reducing agent in dissolved or suspended form and drying the mixed solution to give the dry mixture.

10. The process of claim 19, wherein the vanadium source is selected from the group consisting of divanadium pentoxide and ammonium vanadate.

11. The process of claim 19, wherein the source of the metal M is selected from the group consisting of nitrates, carboxylates, carbonates, hydrogencarbonates, basic carbonates, oxides, hydroxides and oxide hydroxides of the metal M.

12. The process of claim 19, wherein the phosphate source is formed at least partly by phosphorous acid or hypophosphorous acid.

13. The process of claim 19, wherein the drying to give the dry mixture is effected by spray-drying.

14. A gas phase oxidation catalyst comprising the polynary metal oxide phosphate according to claim 1, wherein M may also be V.

15. The catalyst according to claim 14, comprising a first phase and a second phase in the form of three-dimensional delimited regions, the first phase comprising a catalytically active material based on vanadyl pyrophosphate and the second phase comprising a the polynary metal oxide phosphate according to claim 1.

16. The catalyst according to claim 15, wherein finely divided particles of the second phase are dispersed in the first phase.

17. A process for partial gas phase oxidation or ammoxidation comprises contacting a gas stream which comprises a hydrocarbon and molecular oxygen with the catalyst according to claim 14.

18. The process of claim 17 for preparing maleic anhydride, wherein the hydrocarbon comprises at least four carbon atoms.

19. The method of claim 7, wherein the dry mixture further comprises a source of the metal M.

20. The method of claim 19, further comprising providing reduction equivalents to convert either one or both of the vanadium and/or the metal M to a valency state possessed by the vanadium and the metal M in the formula I before the calcining step.

21. The catalyst according to claim 16, wherein the first phase and the second phase are distributed relative to one another as in a mixture of finely divided first phase and finely divided second phase
